(19) United States
(12) Patent Application Publication
(10) Pub. No.: US 2009/0233281 A1
(43) Pub. Date: Sep. 17, 2009

(54) DIAGNOSTIC MARKER FOR ALLERGY

(75) Inventors: Kenichi Masuda, Kanagawa (JP); Yasuyuki Ishii, Kanagawa (JP); Masahiro Sakaguchi, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi, Saitama (JP)

(21) Appl. No.: 12/097,002

(22) PCT Filed: Nov. 15, 2006
(86) PCT No.: PCT/JP2006/322732

§ 371 Date: Aug. 1, 2008

(30) Foreign Application Priority Data
Dec. 12, 2005 (JP) 2005-358327
Jun. 6, 2006 (JP) 2006-157690

(51) Int. Cl. C12Q 1/68; C07K 16/00; C07H 21/00; C12Q 1/02; G01N 33/68; C12N 5/06

(52) U.S. Cl. 435/6; 530/387.1; 536/24.31; 536/24.33; 435/29; 436/86; 435/325

(57) ABSTRACT

The present invention provides a test system that specifically detects allergy, a test system that highly sensitively reflects aggravation of allergy and the like.

More particularly, the present invention provides a diagnostic reagent, particularly a diagnostic reagent for allergy, or a reagent for detecting a mast cell and/or a blastogenic T cell, comprising one or more means for measuring CHD2, which is/are selected from the group consisting of a primer, a nucleic acid probe and an antibody; a test method for allergy, comprising measuring CHD2 expression in a biological sample obtained from an animal and evaluating allergy in the animal; a method for detecting a mast cell and/or a blastogenic T cell; and the like.

### TECHNICAL FIELD

The present invention provides a test method, a reagent such as a diagnostic reagent and the like, a pharmaceutical agent, a food, a screening method and the like for allergy and the like.

### BACKGROUND ART

With the increasing number of patients, allergy is becoming a serious social problem in recent years. There are high social needs for the development of its diagnosis and for a suitable treatment method based thereon. For diagnosis of allergy, sensitized antigen has been mainly identified (test of allergic response to what allergen). Such test includes intradermal response or prick test, and serum IgE test that determines antigen specific IgE in the serum. The intradermal reaction and the prick test are test methods in which a cedar pollen antigen solution and the like are intradermally injected or applied to a wound on the skin of the patients and the development of wheal (swollen skin at the injection site) is examined, based on which substances causing allergic response are determined. In the serum IgE test, the substance causing production of blood IgE is examined. The substance causing production of blood IgE is considered to be a substance causing allergic responses in the body. Thus, a substance responsive to IgE is judged to be an allergen.

In this way, conventional allergy tests are useful for finding a substance to be an allergen. However, a substance judged positive is merely considered to have a possibility of causing an allergic response. Whether it actually causes an allergic condition is unknown. Moreover, such test results simply show acquisition of allergy in the past, and problematically do not teach whether the allergic response to the substance is ongoing at present. Accordingly, conventional allergy tests are ineffective for discrimination of allergy from infections (cold, influenza and the like) showing similar symptoms, and further, for judgment whether allergic responses are actually taking place. As the situation stands, these judgments are made by experienced doctors in the clinical situations and objective judgments are not available. Therefore, there is a demand for the development of a test system that senses only the allergy actually taking place (test for specifically detecting allergy), and further, a test system that highly sensitively reflects aggravation of allergy.

In the meantime, as chromatin helicase DNA binding protein 2 (chromodomain helicase DNA binding protein 2: CHD2), for example, one derived from human is registered under GenBank Accession No.: NM_{—}001271. However, there is no scientifically meaningful report on CHD2.

### DISCLOSURE OF THE INVENTION

### Problems to be Solved by the Invention

The present invention aims to provide a test system that specifically detects allergy, and further, a test system that highly sensitively reflects aggravation of allergy and the like.

### Means of Solving the Problems

The present inventors have conducted intensive studies and found that CHD2 is specifically expressed in a mast cell and a blastogenic T cell (same as activated T cell in the present specification), which are the main cells causing allergy. Since the mast cell and blastogenic T cell are remarkably seen in allergic lesion, it is considered that observation of expression of CHD2 enables detection of allergic response involving the mast cell and blastogenic T cell. Accordingly, a test system of CHD2 expression can be useful for allergy diagnosis and as a monitoring index.

The present inventors have also found that a substance controlling the expression or function of CHD2 can be useful as a pharmaceutical agent, a reagent or a food, particularly, for the prophylaxis or treatment of disease such as allergic diseases, immune diseases and the like, or control of function, differentiation or growth of mast cells and T cells (e.g., blastogenic T cells), and that screening for a substance controlling expression or function of CHD2 can be useful for the development of a pharmaceutical agent, a reagent or a food, for example, for the development of a drug for the prophylaxis or treatment of diseases such as allergic disease, immune disease and the like, or a substance capable of controlling the function, differentiation or growth of mast cell or T cell, and the like.

Based on the above-mentioned findings, the present inventors have completed the present invention.

Accordingly, the present invention provides the following invention and the like.

[1] A diagnostic reagent comprising one or more means for measuring CHD2, which is/are selected from the group consisting of a primer, a nucleic acid probe and an antibody.
[2] The reagent of the above-mentioned [1], which is a diagnostic reagent for allergy.
[3] The reagent of the above-mentioned [1], which is a reagent for diagnosing the presence or absence or severity of allergy.
[4] The reagent of the above-mentioned [1], which is a diagnostic reagent for human or dog.
[5] A reagent for detecting a mast cell and/or a blastogenic T cell, comprising one or more means for measuring CHD2, which is/are selected from the group consisting of a primer, a nucleic acid probe and an antibody.
[6] A method for testing an animal for allergy, comprising measuring CHD2 expression in a biological sample obtained from the animal and evaluating the animal for allergy.
[7] A method of detecting a mast cell and/or a blastogenic T cell, comprising measuring CHD2 expression in a sample comprising the mast cell and/or blastogenic T cell.
[8] A pharmaceutical agent, reagent or food, comprising a substance that controls expression or function of CHD2.
[9] A drug for the prophylaxis or treatment of an allergic disease, immune disease or parasitic disease, comprising a substance that controls expression or function of CHD2.
[10] An agent for controlling the function of a mast cell and/or a T cell, comprising a substance that controls expression or function of CHD2.
[11] A method of screening for a candidate effective as a pharmaceutical agent, reagent or food, comprising evaluating whether or not a test substance controls expression or function of CHD2.
[12] A method of screening for a substance capable of preventing or treating an allergic disease, an immune disease or a parasitic disease, comprising evaluating whether or not a test substance controls expression or function of CHD2.
[13] A method of screening for a substance capable of controlling the function of a mast cell and/or a T cell, comprising evaluating whether or not a test substance controls expression or function of CHD2.
[14] A substance, cell or animal selected from the group consisting of the following:
(a) a polypeptide or a partial peptide thereof having not less than 70% amino acid homology to the amino acid sequence shown by SEQ ID NO: 2,
(b) a polynucleotide or partial nucleotide thereof consisting of a nucleotide sequence that hybridizes to a sequence complementary to the nucleotide sequence shown by SEQ ID NO: 1 under highly stringent conditions;
(c) an expression vector of (a);
(d) a transformant containing the expression vector of (c);
(e) an antibody to CHD2 protein or a partial peptide thereof;
(f) a hybridoma that produces a monoclonal antibody to CHD2 protein or a partial peptide thereof;
(g) an antisense nucleic acid, a ribozyme, an RNAi-inducible nucleic acid, a targeting vector, a set of two or more primers or a nucleic acid probe to CHD2; and
(h) a cell or animal wherein CHD2 expression is controlled.

### BRIEF DESCRIPTION OF THE DRAWINGS

FIG. 1 shows CHD2 expression in various organs of mouse.

FIG. 2 shows CHD2 expression in mouse mast cells.

FIG. 3 shows CHD2 expression in mouse T cells and B cells.

The present invention provides a test (or diagnosis) method of pathology or disease such as allergy and the like.

In one embodiment, the test method of the present invention can comprise, for example, (a) measuring the expression of CHD2 in a biological sample taken from an animal, and (b) evaluating the animal for allergy.

CHD2 (chromodomain helicase DNA binding protein 2) belongs to a protein family characterized by a chromatin organization modifier domain and a SNF2 related helicase/ATPase domain, and is assumed to influence DNA transcription by regulating chromatin structure. As sequence information of CHD2, the information derived from various animals are registered in GenBank. For example, human CHD2 is registered as GenBank Accession No. NM_{—}001271, mouse CHD2 is registered as GenBank Accession No. XM_{—}145698, dog CHD2 is registered as GenBank Accession Nos. XM_{—}854029 and XM_{—}536179, rat CHD2 is registered as GenBank Accession No. XM_{—}218790, and chimpanzee CHD2 is registered as GenBank Accession No.:XM_{—}510607.

In the above-mentioned (a), the animal from which the biological sample can be taken can be any animal as long as it can have an allergy. Examples of the animals to which the present invention can be applied include primates, mammals such as rodents, or companion animals, house animals and working animals. More specifically, examples of the animals to which the present invention can be applied include human, monkey, chimpanzee, dog, cat, horse, bovine, swine, sheep, goat, mouse, rat, guinea pig, hamster, rabbit, camel and lama.

The biological sample that can be used in the present invention can be a biological sample taken from the aforementioned animal, i.e., an animal suspected and/or possible to have an allergy. Since expression of CHD2 is not found in tissues or organs adjacent to the external environment, such as skin, lung and gastrointestinal tract, when the expression of CHD2 is found in such tissue or organ, it is considered that mast cells or blastogenic T cells have infiltrated thereinto to cause an allergic response. Accordingly, the biological sample can be a sample derived from such tissue or organ wherein the expression of CHD2 is not generally found. Examples thereof include samples derived from a tissue or an organ such as nasal mucosa, oral mucosa, skin, eye (e.g., conjunctiva, cornea), skin, trachea, lung, gastrointestinal tract, bladder, vaginal mucosa, urethra, earwax (auditory meatus), and mucus (e.g., sputum and the like). From the viewpoint of lower invasiveness, among the above-mentioned samples, samples derived from a tissue such as nasal mucosa, oral mucosa, skin, eye, vaginal mucosa, earwax or mucus (e.g., sputum and the like) are preferable.

The allergy that can be a target of the test method of the present invention is not particularly limited as long as it is caused by a substance or a composition which can be exposed to a living organism, ingested by a living organism, or applied to a living organism. Examples of such substance and composition include pollens (e.g., cedar, cypress, ragweed, white birch, pasture grasses), foods (e.g., milk, buckwheat, egg, peanut, crab, shrimp, fruits, sesame, buckwheat, wheat, rice, corn, meats or meat substitutes), living organisms other than human or materials derived therefrom (e.g., tick, cockroach, or body hair, feces, urine and dandruff of animals such as dog, cat and rabbit, or of birds such as parrot and parakeet), drugs (e.g., antibiotics such as penicillin, anti-inflammatory drugs such as aspirin, inhalation anaesthetics such as sevoflurane, skin medicines for external use), medical supplies (e.g., bronchial lavage fluids for asthma patients, dental tools), livingwares (e.g., cosmetics, hair dressings, creams, detergents, paints, pesticides, accessories), and other substances or compositions and the like (e.g., latex, formaldehyde, metals such as nickel, resins, sunlight). Such substances or compositions are hereinafter to be abbreviated as allergens as necessary.

The allergy that can be a target of the test method of the present invention can be type I allergy or type IV allergy, which are allergies associated with mast cell and T cell. Accordingly, it is possible to distinguish these allergies from type II and type III allergies.

The expression of CHD2 can be measured by a method known per se with a transcription product of CHD2 gene or CHD2 protein as a subject. For example, the expression level of a transcription product of CHD2 gene can be measured by, after preparing a total RNA from the biological sample, gene amplification methods such as PCR (e.g., RT-PCR, real-time PCR, quantitative PCR), LAMP (Loop-mediated isothermal amplification) (see, for example, WO00/28082) and ICAN (Isothermal and Chimeric primer-initiated Amplification of Nucleic acids) (see, for example, WO00/56877), or northern blotting, microarray and the like. Meanwhile, the expression level of a translation product can be measured by, after preparing an extract from the biological sample, immunological methods. As the immunological methods, for example, enzyme immunoassay (EIA) (e.g., competitive direct ELISA, competitive indirect ELISA, sandwich ELISA), radiation immunoassay (RIA), fluorescence immunoassay (FIA), immunochromatography, luminescence immunoassay, spin immunoassay, western blot, and immunohistochemical staining can be mentioned. As the method other than the above-mentioned methods that enables measurement of the expression of CHD2, for example, mass spectrometry can be mentioned.

In the above-mentioned (b), allergy in the animal can be evaluated based on the measurement results of the expression of CHD2. The allergy in the animal can be evaluated from various viewpoints.

For example, the allergy in the animal can be evaluated from the viewpoint of the presence or absence of an allergic response. When the expression of CHD2 is found in a sample derived from a tissue or an organ where the expression of CHD2 is generally absent, it is considered that mast cells and/or blastogenic T cells have infiltrated into the tissue or the organ. Accordingly, when the expression of CHD2 is confirmed in such tissue or organ, it is considered that an allergic response has actually been provoked. For these reasons, the test method of the present invention is advantageous in that an allergy actually provoked can be detected, diagnostic specificity is high, sensitivity is high and the like.

It is also beneficial to evaluate the presence or absence of allergic response using, as a subject, an animal showing an allergy-like symptom. In this embodiment, in the animal showing an allergy-like symptom, whether or not the symptom is in fact caused by an allergy can be evaluated. In other words, it is possible to distinguish a disease showing an allergy-like symptom from allergy. This is due to the fact that many of the diseases showing an allergy-like symptom do not allow infiltration of mast cells and/or blastogenic T cells into the tissue or organ. The test method of the present invention enables distinction from allergy. The disease showing an allergy-like symptom which can be distinguished from allergy by the test method of the present invention is not particularly limited as long as it does not allow infiltration of mast cells and/or blastogenic T cells into the tissue or organ. Examples thereof include infections (e.g., cold, influenza, mycosis, staphylococcal infection, skin virus infections such as rubella), autoimmune diseases (e.g., collagen disease), foreign substances, tumors, psoriasis, sick building syndromes, and parasitic diseases.

The allergy in an animal can also be evaluated from the viewpoint of the level of allergic response. Since the level of allergic response is thought to possibly correlate with the number of mast cells and/or blastogenic T cells in filtrating into the tissue or organ, as well as the expression level of CHD2 in a biological sample derived from the tissue or organ, it is possible to evaluate the level of allergic response based on the expression level of CHD2. Accordingly, the test method of the present invention is useful for, for example, monitoring of the therapeutic effect of an anti-allergic therapy (e.g., hyposensitization therapy).

In another embodiment, the test method of the present invention is useful for, for example, tests on the development, differentiation and maturation of T cells and mast cells (e.g., test of immunologic diseases, monitoring of immunotherapy and the like).

The present invention also provides a reagent for diagnosis.

The reagent of the present invention can contain, for example, a means for measuring CHD2. While the means for measuring CHD2 is not particularly limited as long as it enables measurement of the expression of CHD2, examples thereof include a primer, a nucleic acid probe and an antibody. The present invention also provides such means per se for measuring CHD2. The reagent of the present invention enables, for example, to conveniently perform the test method of the present invention.

The primer that can be contained in the reagent of the present invention is not particularly limited as long as it enables amplification and detection of CHD2 gene. The size of the primer can be, for example, not less than at least about 12 bp, preferably about 15-100 bp, more preferably about 16-50 bp, and even more preferably about 18-35 bp. While the number of the primers that can be contained in the reagent of the present invention is not particularly limited since the necessary number of the primers varies depending on the kind of the method of gene amplification, the reagent of the present invention can contain, for example, two or more primers. The two or more primers may or may not be preliminarily mixed. Such primers can be prepared by a method known per se.

The nucleic acid probe that can be contained in the reagent of the present invention is not particularly limited as long as it enables detection of CHD2 gene. While the nucleic acid probe may be any of DNA and RNA, considering the stability and the like, DNA is preferred. Also, the nucleic acid probe may be any of a single-stranded probe and a double-stranded probe. While the size of the nucleic acid probe is not particularly limited as long as it permits specific hybridization to a transcription product of CHD2 gene, the size is, for example, not less than about 15 bp, preferably about 15-1000 bp, and more preferably about 50-500 bp. The nucleic acid probe may be provided in the form of being immobilized on a substrate like microarray. Such nucleic acid probe can be prepared by a method known per se.

The antibody which can be contained in the reagent of the present invention is not particularly limited as long as it is an antibody capable of specifically binding to CHD2 protein. For example, the antibody against CHD2 protein may be any of a polyclonal antibody and a monoclonal antibody. Also, the antibody may be a fragment of an antibody (e.g., Fab, F(ab′)₂), or a recombinant antibody (e.g., scFv). The antibody may be provided in the form of being immobilized on a substrate such as a plate. The antibody can be prepared by a method known per se.

The polyclonal antibody can be acquired by, for example, subcutaneously or intraperitoneally administering CHD2 protein or a partial peptide thereof (as required, may be prepared as a complex crosslinked to a carrier protein such as bovine serum albumin or KLH (keyhole limpet hemocyanin)) as the antigen, along with a commercially available adjuvant (e.g., Freund's complete or incomplete adjuvant), to an animal about 2 to 4 times at 2 to 3 week intervals (the antibody titer of partially drawn serum should be determined by a known antigen-antibody reaction and its elevation should be confirmed in advance), collecting whole blood about 3 to about 10 days after final immunization, and purifying the antiserum. As the animal to receive the antigen, mammals such as rats, mice, rabbits, goats, guinea pigs, and hamsters can be mentioned.

The monoclonal antibody can be prepared by, for example, a cell fusion method (e.g., Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pages 2-14, Science Forum Shuppan, 1985). For example, the factor is administered subcutaneously or intraperitoneally along with a commercially available adjuvant to a mouse 2 to 4 times, and at about 3 days after the final administration, the spleen or lymph nodes are collected, and leukocytes are collected. These leukocytes and myeloma cells (e.g., NS-1, P3X63Ag8 and the like) are cell-fused to give a hybridoma that produces a monoclonal antibody against the factor. This cell fusion may be performed by the PEG method [J. Immunol. Methods, 81(2): 223-228 (1985)], or by the voltage pulse method [Hybridoma, 7(6): 627-633 (1988)]. A hybridoma that produces the desired monoclonal antibody can be selected by detecting an antibody that binds specifically to the antigen from the culture supernatant using a widely known EIA or RIA method and the like. Cultivation of the hybridoma that produces the monoclonal antibody can be performed in vitro, or in vivo such as in mouse or rat ascitic fluid, preferably in mouse ascitic fluid, and the antibody can be acquired from the culture supernatant of the hybridoma and the ascitic fluid of the animal, respectively.

Where necessary, the means for measuring CHD2 can be provided in the form of being labeled with a labeling substance. Examples of the labeling substance include fluorescent substances such as FITC and FAM, luminescent substances such as luminol, luciferin and lucigenin, radioisotopes such as ³H, ¹⁴C, ³²P, ³⁵S and ¹²³I, affinity substances such as biotin and streptavidin, and the like.

The reagent of the present invention may be provided in the form of a kit comprising a further component in addition to the means for measuring CHD2. In this case, each of the components comprised in the kit can be provided in the form separated from each other, for example, a form wherein each of the components is stored in a different container. For example, when the means for measuring CHD2 is not labeled with a labeling substance, such kit can further comprise a labeling substance. Such kit can also comprise a positive control (e.g., polynucleotide encoding CHD2 gene or a partial nucleotide thereof, CHD2 protein or a partial peptide thereof).

More specifically, when the reagent of the present invention is provided in the form of a kit, the kit can further comprise a component according to the kind of the means employed for measuring CHD2. For example, when the means for measuring CHD2 is a primer, the kit can further comprise a reverse transcriptase and a nucleic acid extract. When the means for measuring CHD2 is a nucleic acid probe, the kit may further comprise a nucleic acid extract. When the means for measuring CHD2 is an antibody, the kit may further comprise a secondary antibody (e.g., anti-IgG antibody) and a reagent for detecting the secondary antibody.

Additionally, when the reagent of the present invention is provided in a kit form, the kit can further comprise a means for collecting a biological sample from an animal. The means for collecting a biological sample from an animal is not particularly limited as long as it enables acquisition of a biological sample from an animal. Examples thereof include non-biopsy instruments (e.g., cell collection devices such as cotton swab, surgical knife and tape, or collection devices for internal liquid of a blister and a pustula (e.g., injection needle)), biopsy instruments (e.g., biopsy needle) and the like.

The present invention provides a detection method for a mast cell and/or a blastogenic T cell.

The detection method of the present invention can comprise, for example, measurement of the expression of CHD2 in a sample containing a mast cell and/or a blastogenic T cell. The detection method of the present invention can be performed in vitro, for example, according to the aforementioned test method. The detection method of the present invention is useful, for example, for the aforementioned test method.

The detection method of the present invention is also useful for screening for a substance or a composition (e.g., food, drink) incapable or capable of inducing allergy, and enables development of a substance or a composition incapable of inducing allergy, or determination of a substance or a composition capable of inducing allergy. In this case, as the sample containing a mast cell and/or a blastogenic T cell, a biological sample derived from the aforementioned animal, which has been exposed to a test substance, has ingested a test substance, or has been applied with a test substance (biological sample a), can be used. In this case, the test substance is not particularly limited as long as it is a substance or a composition which can be exposed to a living organism, ingested by a living organism, or applied (e.g., administration) to a living organism. In this embodiment, following measurement of the expression of CHD2 in biological sample a, the detection method of the present invention can comprise evaluating whether or not the test substance can induce allergy. As the test substance, for example, the substances mentioned below can be used.

Furthermore, the detection method of the present invention is useful for screening for a substance or a composition (e.g., food, drink) capable of suppressing allergy, and enables development of a substance or a composition capable of suppressing allergy. In this case, as the sample containing a mast cell and/or a blastogenic T cell, a biological sample derived from the aforementioned animal, which has been exposed to an allergen, has ingested an allergen, or has been applied with an allergen, and has been exposed to a test substance, has ingested a test substance, or has been applied with a test substance, (biological sample b), can be used. In this case, the test substance is not particularly limited as long as it is a substance or a composition which can be exposed to a living organism, ingested by a living organism, or applied to a living organism. In this embodiment, following measurement of the expression of CHD2 in biological sample b, the detection method of the present invention can comprise evaluating whether or not the test substance can suppress allergy that can be induce by the allergen. As the test substance, for example, the substances mentioned below can be used.

The present invention also provides a reagent for detection of a mast cell and/or a blastogenic T cell. The reagent of the present invention can contain one or more means for measuring CHD2 mentioned above. The reagent for detection of the present invention can be prepared and used, for example, according to the aforementioned reagent for diagnosis. Also, the reagent for detection of the present invention enables to conduct the detection method of the present invention conveniently.

The present invention provides an agent (or a composition) comprising a substance regulating the expression or the function of CHD2. The present invention also provides a substance regulating the expression or the function of CHD2 per se. While there has been no report on the biological role of CHD2, the present inventors have found that CHD2 can play an important role in mast cells and T cells (e.g., blastogenic T cells) from the fact that it is specifically expressed in mast cells and blastogenic T cells.

In one embodiment, the substance regulating the expression or the function of CHD2 can be a substance promoting the expression of CHD2. When used herein, the promotion of the expression of CHD2 includes supply of CHD2 (protein) per se. Examples of the substance promoting the expression of CHD2 include CHD2 (protein), expression vectors comprising a nucleic acid encoding CHD2 (CHD2 expression vectors) and low-molecular-weight compounds.

CHD2 that can be contained in the agent of the present invention can be natural CHD2 derived from the aforementioned animal or a variant thereof, preferably natural CHD2 derived from human or a variant thereof. The amino acid sequence encoded by CHD2 (e.g., amino acid sequence encoded by nucleotide sequence shown by SEQ ID NO: 1, or amino acid sequence shown by SEQ ID NO: 2) may contain one or more mutations in the amino acids (e.g., deletion, substitution, addition, insertion), as long as CHD2 can control (e.g., promote or suppress) function, differentiation or growth of mast cell and/or T cell (e.g., blastogenic T cell), or as long as CHD2 can retain the function (e.g., DNA binding ability) of chromatin organization modifier domain or SNF2 related helicase/ATPase domain.

In more detail, CHD2 that can be contained in the agent of the present invention can have amino acid sequence identity of, for example, not less than about 70%, preferably not less than about 80%, more preferably not less than about 90%, even more preferably not less than about 95%, most preferably not less than about 97%, about 98% or about 99%, relative to the amino acid sequence encoded by the nucleotide sequence shown by SEQ ID NO: 1, or the amino acid sequence shown by SEQ ID NO: 2. The identity (%) can be determined by a method known per se. For example, the identity (%) can be determined by the use of a program (e.g., BLAST, FASTA etc.) conventionally used in the pertinent field at the initial setting. In another aspect, the identity (%) can be determined by the use of any algorithm known in the pertinent field, for example, the algorithms of Needleman et al. (1970) (J. Mol. Biol. 48: 444-453), Myers and Miller (CABIOS, 1988, 4: 11-17) and the like. The algorithm of Needleman et al. is incorporated in the GAP program in the GCG software package (available from www.gcg.com), and the identity (%) can be determined by the use of, for example, any of BLOSUM 62 matrix or PAM250 matrix, and gap weight: 16, 14, 12, 10, 8, 6 or 4, and length weight: 1, 2, 3, 4, 5 or 6. In addition, the algorithm of Myers and Miller is incorporated in the ALIGN program which is a part of the GCG sequence alignment software package. When the ALIGN program is utilized for comparison of amino acid sequences, for example, PAM120 weight residue table, gap length penalty 12, gap penalty 4 can be used. While the identity (%) may be any as long as it is determined by any of the above-mentioned method, a method showing the lowest value in calculation can be preferably employed from among the above-mentioned methods. The partial peptide of CHD2 is not particularly limited as long as it has a length capable of showing given usefulness (e.g., immunogenicity) and, for example, can consist of at least not less than about 8, preferably not less than about 10, more preferably not less than about 12 continuous amino acids.

CHD2 that can be contained in the agent of the present invention may be a protein recoverable from natural CHD2 expressing cells, or a recombinant protein. CHD2 can be prepared by a method known per se and, for example, a) CHD2 may be recovered from a natural CHD2 expressing cell, b) a CHD2 expression vector (described later) may be introduced into a host cell (e.g., genus Escherichia, genus Bacillus, yeast, insect cell, insect, animal cell) to give a transformant, and CHD2 produced by the transformant may be recovered, or c) CHD2 may be synthesized by a cell-free system using rabbit reticulocyte lysate, wheat germ lysate, Escherichia coli lysate and the like. CHD2 is appropriately purified by a method utilizing solubility such as salting out, solvent precipitation and the like; a method mainly utilizing difference in the molecular weight such as dialysis, ultrafiltration, gel filtration, SDS-polyacrylamide gel electrophoresis and the like; a method utilizing difference in the electric charge such as ion exchange chromatography and the like; a method utilizing specific affinity such as affinity chromatography, use of CHD2 antibody and the like; a method utilizing difference in the hydrophobicity such as reversed-phase high performance liquid chromatography and the like; a method utilizing difference in the isoelectric point such as isoelectric focusing and the like; a combination of these methods and the like.

A polynucleotide encoding CHD2, which is inserted into a CHD2 expression vector that can be contained in the agent of the present invention has a nucleotide sequence encoding the above-mentioned amino acid sequence. The above-mentioned polynucleotide to be inserted into the CHD2 expression vector can also be a polynucleotide capable of hybridizing to the nucleotide sequence shown by SEQ ID NO: 1 under highly stringent conditions. The hybridization conditions under highly stringent conditions can be set in reference to already reported conditions (Current Protocols in Molecular Biology, John Wiley & Sons, 6.3.1-6.3.6, 1999). For example, as the hybridization conditions under highly stringent conditions, hybridization using 6×SSC (sodium chloride/sodium citrate)/45° C., then washing (at least once) with 0.2×SSC/0.1% SDS/50-65° C. can be mentioned. A partial nucleotide of polynucleotide encoding CHD2 can be, for example, at least about 15 or 16, preferably about not less than 18, more preferably about not less than 20, in length.

In another embodiment, a substance controlling the expression or function of CHD2 can be a substance suppressing CHD2 expression. As the substance suppressing CHD2 expression, for example, antisense nucleic acid, ribozyme, RNAi inducible nucleic acid (e.g., siRNA), decoy nucleic acid that mimics the region to be bound with a transcription activation factor of CHD2, targeting vector (e.g., vector comprising first and second polynucleotides homologous to CHD2 gene capable of inducing homologous recombination of CHD2 gene, and where necessary, selection marker), and low-molecular-weight compound can be mentioned.

In another embodiment, a substance controlling the expression or function of CHD2 can be a substance suppressing CHD2 function. The substance suppressing CHD2 function is not particularly limited as long as it can prevent the CHD2 action and, for example, CHD2 inhibitory protein (e.g., CHD2 dominant-negative mutant, the aforementioned CHD2 antibody, or chimeric antibody, humanized antibody or human antibody), and expression vector comprising nucleic acid encoding them can be mentioned.

When the substance controlling the expression or function of CHD2 is a nucleic acid molecule or a protein molecule, the agent of the present invention may contain an expression vector comprising, as an active ingredient, a nucleic acid molecule or nucleic acid molecule encoding the protein molecule. In the expression vector, oligonucleotide or polynucleotide encoding the above-mentioned nucleic acid molecule must be functionally linked to a promoter capable of exhibiting a promoter activity in the cell of a mammal to be the administration subject. The promoter to be used is not particularly limited as long as it can function in the administration subject and, for example, virus promoters such as SV40-derived initial promoter, cytomegalo virus LTR, Rous sarcoma virus LTR, MoMuLV-derived LTR, adenovirus-derived initial promoter and the like, as well as promoters of mammal constitutive protein gene such as β-actin gene promoter, PGK gene promoter, transferrin gene promoter, and the like can be mentioned. As the promoter to be used, a promoter specific to a CHD2 expressing cell (e.g., mast cell, T cell such as blastogenic T cell) may be used. Such a promoter may be a promoter of any gene that shows CHD2 expressing cell-specific expression and, for example, c-kit, FcεRIa and FcεRIb can be used as mast cell-specific promoters, and IL-2, OX-40 and Sprouty can be used as T cell (e.g. blastogenic T cell etc.)—specific promoters. The present invention also provides a expression vector comprising such a promoter.

The expression vector preferably contains a transcription termination signal, namely a terminator region, at the downstream of oligo(poly)nucleotide encoding a nucleic acid molecule. In addition, the vector can further contain selection marker genes for selection of transformed cells (gene imparting resistance to pharmaceutical agents such as tetracycline, ampicillin, kanamycin, hygromycin, phosphinothricin, gene complementing auxotrophic mutation and the like).

The vector having a basic structure and used as an expression vector may be a plasmid or a virus vector. Examples of preferable vectors for administration to mammals such as human include virus vectors such as adenovirus, retrovirus, adeno-associated virus, herpesvirus, vaccinia virus, pox virus, polio virus, Sindbis virus, Sendai virus, lentivirus and the like.

The agent of the present invention can contain any carrier such as a pharmaceutically acceptable carrier, in addition to a substance controlling the expression or function of CHD2. Examples of the pharmaceutically acceptable carrier include, but are not limited to, excipients such as sucrose, starch, mannit, sorbit, lactose, glucose, cellulose, talc, calcium phosphate, calcium carbonate and the like; binders such as cellulose, methylcellulose, hydroxypropylcellulose, polypropylpyrrolidone, gelatin, gum arabic, polyethylene glycol, sucrose, starch and the like; disintegrants such as starch, carboxymethylcellulose, hydroxypropyl starch, sodium-glycol-starch, sodium hydrogen carbonate, calcium phosphate, calcium citrate and the like; lubricants such as magnesium stearate, AEROSIL, talc, sodium lauryl sulfate and the like; aromatics such as citric acid, menthol, glycyllysine.ammonium salt, glycine, orange powder and the like; preservatives such as sodium benzoate, sodium bisulfite, methylparaben, propylparaben and the like; stabilizers such as citric acid, sodium citrate, acetic acid and the like; suspensions such as methylcellulose, polyvinylpyrrolidone, aluminum stearate and the like; dispersing agents such as surfactant and the like; diluents such as water, saline, orange juice and the like; base waxes such as cacao butter, polyethylene glycol, white kerosene and the like; and the like.

A preparation suitable for oral administration includes liquid agents obtained by dissolving an effective amount of a substance in a diluting solution such as water and salin; capsule, sachet or tablet containing an effective amount of a substance in the form of a solid or granule, suspension obtained by suspending an effective amount of a substance in a suitable dispersing medium, emulsion obtained by dispersing and emulsifying a solution containing an effective amount of a substance in a suitable dispersing medium; powder; granule and the like.

A preparation suitable for parenteral administration (e.g., intravenous injection, subcutaneous injection, intramuscular injection, topical injecting and the like) includes aqueous and non-aqueous, isotonic sterile solutions for injection, which can contain anti-oxidants, buffers, bacteriostats, isotonicity agents and the like, as well as aqueous and non-aqueous sterile suspensions that can contain suspending agents, solubilizers, thickening agents, stabilizers, preservatives and the like. The preparation can be sealed in a unit-dose or multi-dose container, such as ampoule and vial. In addition, an active ingredient and a pharmaceutically acceptable carrier may be freeze-dried and stored in a state only requiring dissolution or suspending in an appropriate-sterile vehicle immediately before use.

The dose of the agent of the present invention varies depending on the activity and kind of the active ingredient, administration route (e.g., oral, parenteral), severity of the disease, animal species to be the subject of administration, drug acceptability, body weight, age of the subject of administration, and the like, and cannot be generalized. However, the daily dose is generally about 0.001 mg-about 2.0 g per adult human as the amount of the active ingredient.

The agent of the present invention is useful as a pharmaceutical agent, a reagent or a food. For example, the agent of the present invention can be used for the prophylaxis or treatment of immune diseases (e.g., autoimmune disease, immunodeficiency disease), allergic diseases (e.g., cedar pollinosis, asthma, atopic dermatitis), parasitic diseases (e.g., anisakiasis, scab), functional regulation of mast cell and/or T cell (e.g., blastogenic T cell), or regulation of differentiation or growth thereof.

The present invention provides a method of screening for a candidate effective as a pharmaceutical agent, a reagent or a food, or a substance capable of controlling the function, differentiation or growth of a mast cell and/or a T cell (e.g., blastogenic T cell), as well as a substance obtained by the screening method, and an agent (or composition) containing the substance, which comprises evaluating whether or not the test substance can control the expression or function of CHD2.

The test substance to be applied to a screening method may be any compound or composition. Examples thereof include nucleic acids (e.g., nucleoside, oligonucleotide, polynucleotide), carbohydrates (e.g., monosaccharide, disaccharide, oligosaccharide, polysaccharide), lipids (e.g., fatty acid containing saturated or unsaturated, straight chain, branched chain and/or ring), amino acids, proteins (e.g., oligopeptide, polypeptide), organic low-molecular-weight compounds, compound library created using the combinatorial chemistry technique, solid phase synthesis, random peptide library created by the phage display method, natural components (e.g., component derived from microorganism, animal and plant, marine organism), food, drinkable water and the like.

The screening method of the present invention can be performed in any form as long as it can evaluate whether or not the test substance can control the expression or function of CHD2. For example, the screening method of the present invention can be performed based on 1) measurement of CHD2 expression using a cell applicable to the measurement of measuring CHD2 expression, 2) measurement of CHD2 function using a reconstitution system applicable to the measurement of CHD2 function, 3) measurement of CHD2 function using a CHD2 expressing cell, 4) measurement of CHD2 expression or function using an animal and the like.

In the above-mentioned 1), the screening method using a cell applicable to the measurement of CHD2 expression can comprise, for example, the following steps (a) to (c):

(a) a step of contacting a test substance with a cell applicable to the measurement of CHD2 expression;
(b) a step of measuring the expression level of CHD2 in the cell contacted with the test substance, and comparing the expression level with the expression level of CHD2 in the control cell without a contact with the test substance; and
(c) a step of selecting a test substance capable of controlling the expression level of CHD2, based on the comparison result of the above-mentioned (b).

In the above-mentioned method, step (a), a test substance is placed under contact conditions with a cell applicable to the measurement of CHD2 expression. The test substance can be contacted, in a medium, with the cell applicable to the measurement of CHD2 expression.

The cell applicable to the measurement of CHD2 expression refers to a cell applicable to the direct or indirect evaluation of the expression level of the CHD2 product (e.g., transcription product, translation product). The cell applicable to direct evaluation of the expression level of the CHD2 product can be a CHD2 expressing cell and, on the other hand, the cell applicable to indirect evaluation of the expression level of the CHD2 product can be a cell applicable to a reporter assay for a regulatory region of CHD2 gene transcription. The cell applicable to the measurement of CHD2 expression can be a cell of the aforementioned animals.

The CHD2 expressing cell is not particularly limited as long as it potentially expresses CHD2. Those of ordinary skill in the art can easily identify such a cell and can use a primary cultured cell, a cell line induced from the primary cultured cell, a commercially available cell line, a cell line available from a cell bank and the like. In addition, a mast cell and blastogenic T cell are also preferably used as CHD2 expressing cells. Furthermore, a cell from a disease animal model of, for example, allergic disease, immune disease, parasitic disease and the like can also be used.

The cell applicable to the reporter assay for a regulatory region of CHD2 gene transcription refers to a cell comprising a regulatory region of CHD2 gene transcription and a reporter gene operably linked to the region. The regulatory region of CHD2 gene transcription region and the reporter gene can be inserted in an expression vector. The regulatory region of CHD2 gene transcription is not particularly limited as long as it can control CHD2 expression and, for example, a region from the transcription initiation site to about 2 kbp upstream of the CHD2 gene, or a region consisting of the base sequence of the above-mentioned region wherein one or more bases have been deleted, substituted or added, and having an ability to control CHD2 transcription, and the like can be mentioned. The reporter gene may be any as long as it encodes a detectable substance or an enzyme producing a detectable substance and, for example, GFP (green fluorescence protein) gene, GUS (1-glucuronidase) gene, LUC (luciferase) gene, CAT (chloramphenicol acetyltransferase) gene and the like can be mentioned.

A cell into which a regulatory region of CHD2 gene transcription and a reporter gene operably linked to the region are introduced is not particularly limited as long as it is applicable to the evaluation of the regulatory function of CHD2 gene transcription, namely, as long as the expression level of the reporter gene can be quantitatively analyzed. However, as the cell to be subjected to the introduction, CHD2 expressing cell is preferable, since it expresses a physiological regulatory factor of CHD2 transcription and is considered to be more appropriate for the evaluation of CHD2 expression regulation.

The medium in which a substance is contacted with a cell applicable to the measurement of CHD2 expression can be appropriately selected according to the type of the cell to be used and the like. Examples of the medium include minimum essential medium (MEM), Dulbecco modified minimum essential medium (DMEM), RPMI1640 medium, 199 medium and the like each containing about 5-20% fetal bovine serum. The culture conditions are also determined appropriately according to the type of the cell to be used and the like. For example, the pH of the medium is about 6-about 8, the culture temperature is generally about 30-about 40° C., and the culture period is about 12-about 72 hr.

In step (b) of the above-mentioned method, the CHD2 expression level in the cell contacted with the test material is first measured. The expression level can be measured by the aforementioned method known per se in consideration of the type of the cell to be used and the like. When a cell applicable to a reporter assay for a regulatory region of CHD2 transcription is used as a cell applicable to the measurement of CHD2 expression, the expression level can be measured based on the signal intensity of the reporter.

Then, the CHb2 expression level in a cell contacted with a test substance is compared with that of a control cell free of a contact with a test substance. The CHD2 expression level is compared preferably based on the presence or absence of a significant difference. The CHD2 expression level in a control cell without contact with a test substance may be measured before or simultaneously with the measurement of the CHD2 expression level in a cell contacted with a test substance. From the aspects of the test precision and reproducibility, simultaneous measurement of the CHD2 expression level is preferable.

In step (c) of the above-mentioned method, a test substance controlling the CHD2 expression level is selected. For example, a test substance increasing the CHD2 expression level (promotion of expression) or decreasing the CHD2 expression level (suppression of expression) is useful, for example, for the prophylaxis or treatment of the aforementioned diseases or control of the function, differentiation or growth of a mast cell and/or a T cell (e.g., blastogenic T cell).

In the above-mentioned 2), the reconstituted system applicable to the measurement of the CHD2 function means no cell culture system applicable to the evaluation of CHD2 function controlling activity of a test substance, which comprises CHD2 (protein) and other factors (e.g., nucleic acid molecule, protein). The screening method of the present invention using a reconstituted system can comprise, for example, the following steps (a) to (c):

(a) a step of contacting a test substance with CHD2 (protein) and a binding factor thereof;
(b) a step of measuring the amount of complexes comprising CHD2 and a binding factor thereof, which is contacted with the test substance, and comparing the amount with the amount of complexes without a contact with the test substance; and
(c) a step of selecting a test substance capable of controlling the CHD2 function, based on the comparison result of the above-mentioned (b).

In step (a) of the above-mentioned method, a test substance, CHD2 and a binding factor thereof are contacted in an assay system permitting formation of a complex containing CHD2 and the binding factor thereof. As the binding factor, for example, DNA can be mentioned. In addition, one or both of CHD2 and the binding factor thereof may be labeled to facilitate detection of the complex. As the labeling, for example, fusion with a protein that can be encoded by a reporter gene can be mentioned in addition to the labeling with the aforementioned labeling substances. In the assay system, moreover, cell homogenates including CHD2 and/or other factors (e.g., binding factor) and the like (e.g., homogenate of cell transfected with a CHD2 expression vector and/or CHD2 binding factor expressing vector) can also be used.

In step (b) of the above-mentioned method, the amount of complexes contacted with a test substance is first measured. The amount of such complexes can be measured by a method known per se, for example, immunological methods (e.g., immunoprecipitation method, ELISA), and interaction analysis methods utilizing surface plasmon resonance (e.g., use of Biacore™) can be mentioned.

Then, the amount of complexes contacted with a test substance is compared with the amount of complex without a contact with a test substance. The amounts of complexes are compared preferably based on the presence or absence of a significant difference. The amount of complex without a contact with a test substance may be measured before or simultaneously with the measurement of the amount of complexes contacted with a test substance. From the aspects of the test precision and reproducibility, simultaneous measurement of the amount of complex is preferable.

In step (c) of the above-mentioned method, a test substance controlling the amount of complex is selected. For example, a test substance increasing the amount of complexes (promotion of complex formation), or decreasing the amount of complexes (suppression of complex formation) is useful, for example, for the prophylaxis or treatment of the aforementioned diseases or control of the function, differentiation or growth of a mast cell and/or a T cell (e.g., blastogenic T cell).

In the above-mentioned 3), the screening method using a CHD2 expressing cell for the measurement of the functional level of CHD2 can comprise, for example, the following steps (a) to (c):

(a) a step of contacting a test substance with a CHD2 expressing cell;
(b) a step of measuring the functional level of CHD2 in the cell contacted with the test substance, and comparing the functional level with the functional level of CHD2 in the control cell without a contact with the test substance; and
(c) a step of selecting a test substance capable of controlling the functional level of CHD2, based on the comparison result of the above-mentioned (b).

In step (a) of the above-mentioned method, a test substance is placed under contact with a CHD2 expressing cell. A test substance can be contacted, in a medium, with a CHD2 expressing cell. The CHD2 expressing cell to be used here can be a cell capable of expressing CHD2 to the extent that CHD2 expression levels can be assayed at a protein level. Preferable examples of such CHD2 expressing cell include a cell transfected with a CHD2 expression vector and/or an expression vector of a CHD2 binding factor (e.g., mast cell, T cell). A test substance can be contacted, in a medium, with a CHD2 expressing cell.

In step (b) of the above-mentioned method, the CHD2 functional level in a cell contacted with a test substance is first measured. The CHD2 functional level can be measured, for example, by a two-hybrid system besides the method of the above-mentioned 2). The comparison of the functional level in step (b), and the above-mentioned method step (c) can be performed in the same manner as in the above-mentioned method 2).

In the above-mentioned 4), the screening method of the present invention using an animal can comprise, for example, the following steps (a) to (c):

(a) a step of administering a test substance to an animal;
(b) a step of measuring the expression level or functional level of CHD2 in the animal administered with the test substance, and comparing the expression level or functional level with the expression level or functional level of CHD2 in the animal free of administration of the test substance; and
(c) a step of selecting a test substance capable of controlling the expression level or functional level of CHD2, based on the comparison result of the above-mentioned (b).

This methodology can also essentially comprise steps (b) and (c).

As the animal in step (a) of the above-mentioned method, for example, the aforementioned animals can be used. As the animal, the aforementioned disease animal models can be used. A test substance can be administered to the animal by a method known per se.

In step (b) of the above-mentioned method, the expression level or functional level of CHD2 can be measured by a method known per se. For example, the expression level or functional level of CHD2 in a mast cell or a T cell (e.g., blastogenic T cell) isolated or collected from an animal or the expression level or functional level of CHD2 in the aforementioned biological sample can be measured by a methodology similar to that in step (b) of the above-mentioned methods 1)-3). The comparison of the expression level in step (b), and the above-mentioned method step (c) can also be performed by a methodology similar to that in the above-mentioned methods 1)-3).

The present invention provides a cell and an animal wherein the CHD2 expression is controlled. The control of expression can be suppression of expression (e.g., knockout) or promotion of expression (excess or stable expression). The cell and the animal of the present invention can be those wherein CHD2 expression is transiently or constitutively controlled, with preference given to those wherein the expression is constitutively controlled. As the cell of the present invention, mast cells and T cells (e.g., blastogenic T cells) are also preferable, and as the animal of the present invention, one showing expression controlled to be specific to such cell is also preferable. The cell of the present invention can be one derived from the animal of the present invention, or a primary cultured cell or cell line. Such cells and animals can be prepared using a vector described in the present specification (e.g., expression vector such as mast cell and/or T cell (e.g., blastogenic T cell)-specific expression vector and the like, or targeting vector) and the like and according to a method known per se.

The contents disclosed in any publication cited herein, including patents and patent applications, are hereby incorporated in their entireties by reference in the present specification, to the extent that they have been disclosed herein.

The present invention is hereinafter described in more detail by means of the following Examples, which, however, are not to be construed as limiting the invention.

### EXAMPLES

### Materials and Methods

### 1) Preparation of Mouse Bone Marrow-Derived Mast Cells

Mouse bone marrow-derived mast cells were prepared by culturing the femur bone marrow excised from 8-week-old female BALB/c and scid mice in mouse interleukin-3 (5 ng/ml)-added α-MEM medium for 4 to 8 weeks. These cells were collected and their RNA was extracted as templates for RT-PCR. Cells that had been cultured with retinoic acid (ATRA) (1 μM), mouse IL-4 (50 ng/mL) and lipopolysaccharide (10 μg/mL) stimulus for 4 and 14 hr were also collected.

### 2) Separation of Mouse B and T Cells.

The separation of B and T cells was performed by MACS (magnetic cell sorting: a method for separating cells with magnetized antibody). RNA was extracted from some of such cells directly following freezing. The rest cells were collected following stimulation with mouse interleukin-4 (50 ng/ml) and lipopolysaccharide (10 μg/ml) for 4 and 14 hr and their RNA was extracted.

3) RNA Extraction and cDNA Preparation from Mouse-Derived Samples

Various organs were excised from 8-week-old female BALB/c and scid mice. Total RNA was extracted from mast, T and B cells that had been separated and cultured as described above. Fast RNA extraction kit was used for total RNA extraction. Extracted RNA was prepared at the concentration of 20-100 μg/ml. cDNA was prepared by reverse transcriptase. RT reaction for cDNA preparation was performed using GeneAmp RNA PCR Core Kit (Applied Biosystems) at 25° C. for 10 min, at 42° C. for 15 min and at 72° C. for 10 min.

### 4) Primer Design for Mouse CHD2.

Primers were designed using computer software Genetyx (Genetyx Corporation) to detect mouse CHD2 by RT-PCR. Primers were prepared based on a previously reported, predicted mouse CHD2 cDNA sequence 539-557 (forward primer) and 738-719 (reverse primer), the base sequences of which were 5′-acgacgacgatgatgatgaa-3′ (SEQ ID NO: 3) and 5′-ggctcctttctttcccagtc-3′ (SEQ ID NO: 4), respectively.

### 5) PCR Reaction for Mouse CHD2

Using the above-mentioned primer pair and template cDNA, which had been obtained by RT reaction, PCR reaction was performed on a gene amplifier for 40 cycles of 95° C. for 1 min, 55° C. for 1 min and 72° C. for 1 min after denaturation at 95° C. for 5 min. Samples were electrophoresed on 3% agarose gels. Gels were then stained with ethidium bromide. Amplified PCR products were visualized under UV light.

### 6) Separation of Human Peripheral Blood and Isolation of Human T and B Cells

Ten cc of the peripheral blood was collected from a volunteer and diluted to 2-fold with PBS. This sample was then overlaid on Ficoll and centrifuged at 1500 rpm for 40 min to collect mononuclear cell fraction (peripheral blood mononuclear cell, PBMC).

Using MACS (magnetic cell sorting) system (pan T cell Isolation Kit and Bcell Isolation Kit, MiltenYi Biotec), human T and B cells were separately isolated from PMBC according to a conventional method.

Also, peripheral blood-derived cultured mast cells were cultured on methylcellulose containing IL-3 (1 ng/ml), stem cell factor (200 ng/ml) and IL-6 (50 ng/ml) for 6 weeks and were subsequently cultured and maintained in liquid medium containing SCF (100 ng/ml) and IL-6 (50 ng/ml) to permit the selection of the cultured mast cells.

7) RNA Extraction from Human-Derived Samples and PCR

Using an RNA extraction kit, RNeasy Mini Kit (QIAGEN), RNA was extracted from human T, non-T, B, PMBC and mast cells. Extracted RNA was prepared at the concentration of 20-100 μg/ml to prepare cDNA using reverse transcriptase.

RT reaction for cDNA preparation was performed at 25° C. for 10 min, at 42° C. for 15 min, and at 72° C. for 10 min.

Primers were designed using computer software Genetyx (Genetyx Corporation) to detect human CHD2 mRNA by RT-PCR. Primers were prepared based on the previously reported human CHD2 cDNA sequence (AF006514) 648-668 (forward primer) and 853-835 (reverse primer). Their base sequences are 5′-GCACAGGACTTCAAAGCAAAC-3′ (SEQ ID NO: 5) and 5′-TGTCCACTTCCTGGATCAC-3′ (SEQ ID NO: 6), respectively. The size of human CHD2 PCR product is 206 bp. Also, human GAPDH was utilized as an internal standard molecule. The expression analysis of human GAPDH mRNA was performed using a previously reported primer pair (Stefan Bauer, et al., PNAS, 2001; 98 9237-9242) to detect a 157 bp PCR product.

Using the above-mentioned primer pair and template cDNA, which had been obtained by RT reaction, PCR reaction was performed on a gene amplifier for 29 cycles of 95° C. for 30 sec, 62° C. for 1 min and 72° C. for 1.5 min after denaturation at 95° C. for 5 min for human CHD2 mRNA detection. For human CHD2 mRNA detection, PCR reaction was performed on a gene amplifier for 29 cycles of 95° C. for 30 sec, 58° C. for 1 min and 72° C. for 1.5 min after denaturation at 95° C. for 5 min. Samples were electrophoresed on 3% agarose gels. Gels were then stained with ethidium bromide. Amplified PCR products were visualized under UV light. Also, the sequence of this PCR product was analyzed to confirm this to be a human CHD2 mRNA-derived fragment.

8) RNA Extraction from Dog-Derived Samples and Quantitative Real-Time PCR

Collected dog skin samples were homogenated using Tissue Lyser (QIAGEN) for 5 min. RNA extraction was then performed using an RNA extraction kit, RNeasy Mini Kit (QIAGEN). Collected RNA was prepared at the concentration of 50 ng/ml. mRNA expression levels for dog CHD2, other molecules that are expressed upon allergic dermatitis (c-kit and stem cell factor), and an internal standard molecule (GAPDH) were quantitatively measured by TaqMan real-time PCR method using Applied Biosystems 7500 Real-Time PCR System (Applied Biosystems Japan).

Primers and probes used in experiments are as follows (Table 1).

TABLE 1

| molecule | Accession | | |
|---|---|---|---|
| name | No. | position | base sequence |
| CHD2 | XM 536179 | | |
| | Forward | 5257-5274 | CAAGAAGAGGAGGAGCAA (SEQ ID |
| | Primer | | NO: 7) |
| | Reverse | 5352-5371 | GTGGAGTGTGGGACTGAGATA (SEQ |
| | Primer | | ID NO: 8) |
| | QuantiProbe | 5293-5309 | ATTGGTGGTAAGAAGCC (SEQ ID |
| | | | NO: 9) |
| stem cell | NM 001012735 | | |
| factor | Forward | 592-611 | CAGCAGTAGCAGTAATAGGAA (SEQ |
| (SCF) | Primer | | ID NO: 10) |
| | Reverse | 686-704 | GCTCCAAAAGCAAACCCAA (SEQ ID |
| | Primer | | NO: 11) |
| | QuantiProbe | 636-652 | CAACTTACAATGgGCAG (SEQ ID |
| | | | NO: 12) |
| c-kit | NM 001003181 | | |
| | Forward | 1736-1753 | ATAGACCCAACACAGCTTC (SEQ ID |
| | Primer | | NO: 13) |
| | Reverse | 1798-1817 | CAGCACCCAAAGTTTTCCCAA (SEQ |
| | Primer | | ID NO: 14) |
| | QuantiProbe | 1777-1793 | TCCCAGAAACAGGCTGA (SEQ ID |
| | | | NO: 15) |
| GAPDH | NM 001003142 | | |
| | Forward | 739-756 | CTGGAGAAAGCTGCCAAA (SEQ ID |
| | Primer | | NO: 16) |
| | Reverse | 839-856 | TGTTGAAGTCACAGGAGA (SEQ ID |
| | Primer | | NO: 17) |
| | QuantiProbe | 770-786 | AGAAGGTAGTGAAGCAG (SEQ ID |
| | | | NO: 18) |

### Example 1

### CHD2 Expression Levels in Various Mouse Organs

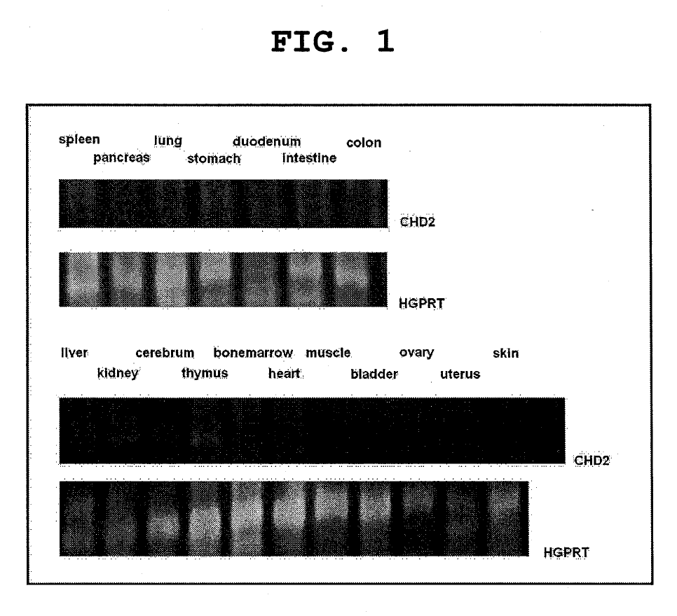

CHD2 expression levels in various mouse organs were measured according to above-mentioned 3)-5). Results are shown in FIG. 1.

As a result, expression was detected only in the liver, kidney, cerebrum and thymus.

### Example 2

### CHD2 Expression in Mouse Mast Cells

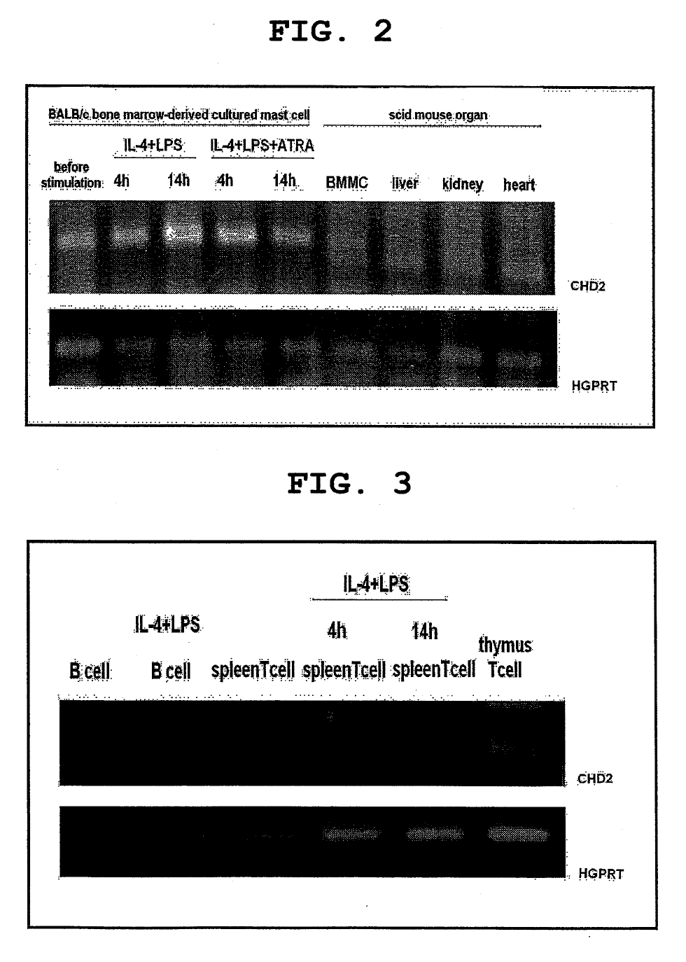

CHD2 expression levels in mouse mast cells were then measured according to above-mentioned 3)-5). Results are shown in FIG. 2.

As a result, CHD2 was highly expressed in mouse bone marrow-derived cultured mast cells (BMMCs). In scid mice that lack T and B cells, however, expression was weakly detected in bone marrow-derived cultured mast cells as well as in the heart, but not in the liver and kidney at all. In addition, tests of CHD2 expression in other animal-derived mast cells resulted in the detection of similar high CHD2 expression levels in a dog-derived mast cell line (data not shown).

As described above, CHD2 was a molecule that was highly expressed in mast cells. Also, liver- and kidney-expressed CHD2 of BALB/c mice was suggested to be derived from their mast cells because there was no detectable CHD2 expression in the liver and kidney of scid mice (lacking B and T cells).

### Example 3

### CHD2 Expression Patterns in Mouse T and B Cells

CHD2 expression patterns in mouse T and B cells were determined according to above-mentioned 2)-5). Results are shown in FIG. 3.

As a result, although there was no detectable CHD2 expression in mouse spleen-derived B and T cells, transient CHD2 expression was detected after spleen T cells were stimulated with interleukin-4 (IL-4) and lipopolysaccharide (LPS) for 4 hr. In addition, CHD2 expression was found to be constitutive in thymus T cells (blastogenic).

As described above, CHD2 was expressed in spleen blastogenic and thymus T cells.

### Example 4

### CHD2 Expression in Human Mast, T and B Cells

CHD2 expression levels in human mast, T and B cells were then measured according to above-mentioned 6) and 7).

As a result, human CHD2 mRNA was highly expressed in mast cells, but CHD2 expression was detected only weakly in T and B cells.

As described above, CHD2 was also useful for the detection of mast cells in humans.

### Example 5

### CHD2 Expression in Blown Nasal Secretions of Humans

Nasal mucous membranes of cedar pollinosis, allergic rhinitis patients were scraped several times using a cotton swab. The cotton swab was then immersed in a cell lysate solution of RNeasy Mini Kit (QIAGEN). RNA was extracted from the lysate solution according to a conventional method. mRNA expression levels of human CHD2 and an internal standard molecule, human GAPDH, were measured by RT-PCR method under similar conditions as above 7). The RNA of human peripheral blood-derived cultured mast cells was utilized as a positive control.

As a result, human CHD2 expression was also detected in blown nasal secretions of cedar pollinosis, allergic rhinitis patients.

As described above, it was possible to diagnose allergy for humans by measuring CHD2 expression levels in human-derived samples.

### Example 6

### CHD2 Expression in Allergic Dermatitis Lesions of Dogs

Skin biopsy samples of cases where dogs had been clinically diagnosed as allergic dermatitis were collected by punch biopsy at 5 mm diameter. CHD2 mRNA expression levels were measured in dog allergic dermatitis lesions using this biopsy samples. RNA extraction and quantitative real-time PCR were performed according to the method as above 8).

As a result, expression levels of CHD2 were low in cases where mast cell infiltration occurred only slightly while they were high in cases where mast cell infiltration extensively occurred, similar to those of SCF and c-kit, which are upregulated at allergic lesions, and thus regarded as indicators of mast cell infiltration: the correlation between CHD2 mRNA expression patterns and SCF and c-kit mRNA expression patterns was recognized.

As described above, CHD2 was also able to be utilized as a marker of mast cell-related allergy in dog allergic dermatitis. In addition, the detection of CHD2 enabled allergy diagnosis not only in medical fields of humans, but also in those of animals.

### INDUSTRIAL APPLICABILITY

A test method of the present invention is useful for a test of the presence or absence or severity of allergy or a test in relation to T and mast cell development, differentiation and maturation (e.g., test of immune disease or monitoring of immunotherapy) and the like.

A detection method of the present invention is useful for an aforementioned test method, the development of substances or compositions that can not induce allergy, the identification of substances or compositions that can induce allergy or the development of substances or compositions that can suppress allergy.

A diagnosis reagent or detection reagent of the present invention is useful for a test method of the present invention or for achieving to simplify a test method or detection method of the present invention.

A pharmaceutical agent, reagent or food of the preset invention is useful as a therapeutic or prophylactic drug for an allergic disease, immune disease or parasitic disease, as a regulator of CHD2 expression or function or as a functional food in relation thereto.

A screening method of the present invention is useful for the development of an above-mentioned pharmaceutical agent, reagent or food.

A polypeptide of the present invention or partial peptide thereof, polynucleotide of the present invention or partial nucleotide thereof, expression vector of the present invention, transformant incorporating the expression vector(s) of the present invention, antibody of the present invention, hybridoma of the present invention, antisense nucleic acid of the present invention, ribozyme of the present invention, RNAi-inducible nucleic acid of the present invention, targeting vector of the present invention, primer set or nucleic acid probe of the present invention, cell of the present invention or animal of the present invention is useful for performing the test or detection method of the present invention or for the development of the diagnosis or detection reagent of the present invention.

The present invention is based on patent applications No. 2005-358327 filed in Japan (filing date: Dec. 12, 2005) and No. 2006-157690 filed in Japan (filing date: Jun. 6, 2006), the contents of which are incorporated in full herein by this reference.

1. A diagnostic reagent comprising one or more means for measuring CHD2, which is/are selected from the group consisting of a primer, a nucleic acid probe and an antibody.

2. The reagent of claim 1, which is a diagnostic reagent for allergy.

3. The reagent of claim 1, which is a reagent for diagnosing the presence or absence or severity of allergy.

4. The reagent of claim 1, which is a diagnostic reagent for human or dog.

5. A reagent for detecting a mast cell and/or a blastogenic T cell, comprising one or more means for measuring CHD2, which is/are selected from the group consisting of a primer, a nucleic acid probe and an antibody.

6. A method for testing an animal for allergy, comprising measuring CHD2 expression in a biological sample obtained from the animal and evaluating the animal for allergy.

7. A method of detecting a mast cell and/or a blastogenic T cell, comprising measuring CHD2 expression in a sample comprising the mast cell and/or blastogenic T cell.

8. A pharmaceutical agent, reagent or food, comprising a substance that controls expression or function of CHD2.

9. A drug for the prophylaxis or treatment of an allergic disease, immune disease or parasitic disease, comprising a substance that controls expression or function of CHD2.

10. An agent for controlling the function of a mast cell and/or a T cell, comprising a substance that controls expression or function of CHD2.

11. A method of screening for a candidate effective as a pharmaceutical agent, reagent or food, comprising evaluating whether or not a test substance controls expression or function of CHD2.

12. A method of screening for a substance capable of preventing or treating an allergic disease, an immune disease or a parasitic disease, comprising evaluating whether or not a test substance controls expression or function of CHD2.

13. A method of screening for a substance capable of controlling the function of a mast cell and/or a T cell, comprising evaluating whether or not a test substance controls expression or function of CHD2.

14. A substance, cell or animal selected from the group consisting of the following:
(a) a polypeptide or a partial peptide thereof having not less than 70% amino acid homology to the amino acid sequence shown by SEQ ID NO: 2;
(b) a polynucleotide or partial nucleotide thereof consisting of a nucleotide sequence that hybridizes to a sequence complementary to the nucleotide sequence shown by SEQ ID NO: 1 under highly stringent conditions;
(c) an expression vector of (a);
(d) a transformant containing the expression vector of (c);
(e) an antibody to CHD2 protein or a partial peptide thereof;
(f) a hybridoma that produces a monoclonal antibody to CHD2 protein or a partial peptide thereof;
(g) an antisense nucleic acid, a ribozyme, an RNAi-inducible nucleic acid, a targeting vector, a set of two or more primers or a nucleic acid probe to CHD2; and
(h) a cell or animal wherein CHD2 expression is controlled.


SEQUENCE LISTING (18 sequences)

SEQ ID NO: 1 - DNA, 5220 bp, Homo sapiens
  CDS (1)..(5220)
atgatgagaaataaggacaaaagccaagaggaggacagttcgctacacagcaatgcatcgagtcactcagcctctgaagaagcttcgggttcagactcaggcagtcagtcggaaagtgagcagggaagtgatccaggaagtggacatggcagcgagtcgaacagcagctctgaatcttctgagagtcagtcggaatctgagagcgaatcagcaggttccaaatcccagccagtcctcccagaagccaaagagaagccagcctctaagaaggaacggatagctgatgtgaagaagatgtgggaagaatatcctgatgtttatggggtcaggcggtcaaaccgaagcagacaagaaccatcgcgatttaatattaaggaagaggcaagtagcgggtctgagagtgggagcccaaaaagaagaggccagaggcagctgaaaaaacaagaaaaatggaaacaggaaccctcagaagatgaacaggaacaaggcaccagtgcagagagtgagccagaacaaaaaaaagtaaaagccagaagacctgtccccagaagaacagtgcccaaacctcgtgttaaaaagcagccgaagactcagcgtggaaagagaaaaaagcaagattcttctgatgaggatgatgatgatgacgaagctcccaaaaggcagactcgtcgaagagcggctaaaaacgttagttacaaagaagatgatgactttgagactgactcagatgatctcattgaaatgactggagaaggagttgatgaacagcaagataatagtgaaactattgaaaaggtcttagattcaagactgggaaagaaaggagccactggagcatctactactgtatatgcgattgaagctaatggcgaccctagtggtgactttgacactgaaaaggatgaaggtgaaatccagtacctcatcaagtggaagggttggtcttacatccacagcacatgggagagtgaagaatccttacagcaacagaaagtgaagggcctaaaaaaactagagaacttcaagaaaaaagaggacgaaatcaaacaatggttagggaaagtttctcctgaagatgtagaatatttcaattgccaacaggagctggcttcagagttgaataaacagtatcagatagtagaaagagtaatagctgtgaagacaagtaaatctacattgggtcaaacagattttccagctcatagtcggaagccggcaccctcaaatgagcccgaatatctatgtaaatggatgggactcccctattcagagtgtagctgggaagatgaagccctcattggaaagaaattccagaattgcattgacagcttccacagtaggaacaactcaaaaaccatcccaacaagagaatgcaaggccctgaagcagagaccacgatttgtagctttaaagaaacaacctgcatatttaggaggggagaatctggaacttcgagattatcagctagaaggtctaaactggctagctcattcctggtgcaaaaataatagtgtaatccttgctgatgaaatgggcctaggaaagaccatccagaccatatcattcctctcctacctgttccaccaacaccagctgtatggcccctttcttatagtcgtccctttatccaccctcacctcatggcagagagagtttgaaatctgggcaccagagattaacgtagtggtttacataggtgacctgatgagcagaaatacgatacgggaatatgaatggattcattcccaaaccaaaagattgaagttcaacgcacttataacaacatatgagatcctcttgaaagataagactgtgctgggcagtattaactgggcctttctgggagtggatgaagcccatcggttgaagaatgatgactctttattgtataaaactctgattgatttcaagtccaaccataggctcctgattacggggacccctcttcagaattccctcaaagagctctggtccttgctgcactttattatgccggagaagtttgaattttgggaagattttgaagaagaccatgggaaggggagagagaatggctaccagagtcttcataaggtgctagagcctttccttctccggagagtcaaaaaagatgtggagaaatcccttcctgctaaagtggaacagattctcagggtggagatgtcagcccttcagaaacagtattacaagtggattctgaccaggaattacaaggctcttgccaaaggaacaagaggcagcacatctggttttcttaatattgtgatggaactgaaaaaatgttgcaaccactgctatctgattaaaccccctgaagaaaatgaaagggaaaatggacaggagattcttctgtccctcataaggagcagtgggaagttgattttattagacaaactgttgacaagacttcgagaaagggggaatcgagtgcttatcttctctcagatggtgagaatgttggatatcctggctgaatacctaactattaaacactatcctttccagcgtctggatggttccatcaagggagaaatccgaaaacaggcactggaccacttcaatgcagatgggtctgaggacttctgtttcctgctctcgacaagggctggtggcctgggaatcaatttggcttcagcggacacagtcgtcatctttgactctgactggaacccccagaatgacttgcaggcacaagcccgagcgcatagaattggtcagaagaagcaggtaaatatttaccgcttagttacaaaggggactgtggaggaggagatcatagaacgggccaaaaagaagatggtattagatcatctggtgattcagcgcatggacaccactggccggacgatcctggaaaacaactcaggaaggtccaactcaaatccttttaataaagaagagctgacagctattttgaaatttggagcagaggatctcttcaaagaactggaaggggaggaatcagaacctcaggaaatggatatagatgaaattttgcggttggctgaaacgagagagaatgaagtgtcaacaagtgcaacagatgaacttctatcacagtttaaggttgccaactttgcaacaatggaagatgaagaagagctagaagagcgtcctcacaaggactgggatgagatcattccagaggaacaaaggaaaaaagtagaggaggaagagcggcagaaggagctagaagaaatttatatgctgcctcgaattcggagttccactaaaaaggctcagacaaatgacagtgactctgacactgagtctaagaggcaggcccagagatcctctgcttctgagagtgaaacggaagactctgatgatgacaagaagccaaagcgcagagggcgtccgaggagtgtgcggaaggacctcgtggagggatttactgatgcagagatccgaaggttcatcaaggcttataagaagtttggtctccctcttgaacggctggagtgcttagcacgtgatgctgagctggtagataagtcggtggcagatctgaagcgcctgggtgaactgatccacaacagctgtgtgtcagcaatgcaggaatatgaagagcagctgaaagaaaatgccagcgagggaaaaggaccagggaaaaggagaggtccaacaatcaagatatccggagttcaggttaatgtgaaatccattatccaacatgaagaggagtttgagatgctgcataaatctatccctgtggaccctgaagaaaaaaaaaaatactgcttaacctgtcgtgtcaaagctgcacattttgatgtagagtggggggtggaagatgattctcgcctgttgctggggatttatgaacatggctatggaaactgggagttaattaaaacagacccagagcttaaattaactgacaaaattctgccggtggagacagataaaaagcctcaggggaagcagctacagacccgagcggattacttgttgaagctgctcagaaagggtctggagaagaagggggctgtgacaggtggggaggaggccaaattaaagaagcggaagcctcgggtaaagaaggaaaacaaagtgcccaggctgaaagaggagcatggaattgagctttcatctcctaggcattcagataatccatcagaagagggagaagtgaaagatgatggcttggaaaaaagtccaatgaaaaaaaaacagaagaagaaagagaacaaggagaacaaggagaaacaaatgagttctaggaaagacaaagaaggggacaaggaaagaaagaagtcaaaagataagaaagagaagcctaaaagtggtgatgccaaatcttcgagtaaatcaaagcgatctcagggtcctgtccatattacagcaggaagtgaacctgtccccattggagaggatgaggatgatgatctggaccaggagacattcagcatatgtaaggagaggatgaggcccgtgaaaaaggcactgaaacagctcgacaaacctgacaaggggctcaacgtgcaagaacagctggaacacacccggaactgcctgctgaaaatcggagaccggatagccgagtgccttaaagcctactcagatcaggagcacatcaaactctggaggaggaacctatggatttttgtttccaagtttacagaatttgatgctcgaaaactgcataagttatacaagatggctcataagaaaaggtctcaagaagaagaggagcaaaagaagaaagacgacgtgactgggggtaagaaaccatttcgtccagaggcctcaggctccagccgggactctctgatatctcagtcccatacctcacacaaccttcaccctcagaagcctcatttgcctgcctcccatggcccacagatgcatggacacccaagagataactacaatcaccccaacaagagacacttcagtaatgcagatcgaggagactggcagagggaaagaaagttcaactatggtggtggcaacaacaatccaccatggggaagcgacaggcaccatcagtatgagcagcactggtacaaggaccaccattatggggaccggcgacatatggatgcccaccgttccggaagctatcgacccaacaacatgtccagaaagaggccttatgaccagtacagcagtgaccgagaccaccggggacacagagattattatgacaggtatgcaaaaggctgtgagacaccaggtgccaacctttgccaggagctgtttctagggagaaagtga

SEQ ID NO: 2 - protein, 1739 aa, Homo sapiens
MMRNKDKSQEEDSSLHSNASSHSASEEASGSDSGSQSESEQGSDPGSGHGSESNSSSESSESQSESESESAGSKSQPVLPEAKEKPASKKERIADVKKMWEEYPDVYGVRRSNRSRQEPSRFNIKEEASSGSESGSPKRRGQRQLKKQEKWKQEPSEDEQEQGTSAESEPEQKKVKARRPVPRRTVPKPRVKKQPKTQRGKRKKQDSSDEDDDDDEAPKRQTRRRAAKNVSYKEDDDFETDSDDLIEMTGEGVDEQQDNSETIEKVLDSRLGKKGATGASTTVYAIEANGDPSGDFDTEKDEGEIQYLIKWKGWSYIHSTWESEESLQQQKVKGLKKLENFKKKEDEIKQWLGKVSPEDVEYFNCQQELASELNKQYQIVERVIAVKTSKSTLGQTDFPAHSRKPAPSNEPEYLCKWMGLPYSECSWEDEALIGKKFQNCIDSFHSRNNSKTIPTRECKALKQRPRFVALKKQPAYLGGENLELRDYQLEGLNWLAHSWCKNNSVILADEMGLGKTIQTISFLSYLFHQHQLYGPFLIVVPLSTLTSWQREFEIWAPEINVVVYIGDLMSRNTIREYEWIHSQTKRLKFNALITTYEILLKDKTVLGSINWAFLGVDEAHRLKNDDSLLYKTLIDFKSNHRLLITGTPLQNSLKELWSLLHFIMPEKFEFWEDFEEDHGKGRENGYQSLHKVLEPFLLRRVKKDVEKSLPAKVEQILRVEMSALQKQYYKWILTRNYKALAKGTRGSTSGFLNIVMELKKCCNHCYLIKPPEENERENGQEILLSLIRSSGKLILLDKLLTRLRERGNRVLIFSQMVRMLDILAEYLTIKHYPFQRLDGSIKGEIRKQALDHFNADGSEDFCFLLSTRAGGLGINLASADTVVIFDSDWNPQNDLQAQARAHRIGQKKQVNIYRLVTKGTVEEEIIERAKKKMVLDHLVIQRMDTTGRTILENNSGRSNSNPFNKEELTAILKFGAEDLFKELEGEESEPQEMDIDEILRLAETRENEVSTSATDELLSQFKVANFATMEDEEELEERPHKDWDEIIPEEQRKKVEEEERQKELEEIYMLPRIRSSTKKAQTNDSDSDTESKRQAQRSSASESETEDSDDDKKPKRRGRPRSVRKDLVEGFTDAEIRRFIKAYKKFGLPLERLECLARDAELVDKSVADLKRLGELIHNSCVSAMQEYEEQLKENASEGKGPGKRRGPTIKISGVQVNVKSIIQHEEEFEMLHKSIPVDPEEKKKYCLTCRVKAAHFDVEWGVEDDSRLLLGIYEHGYGNWELIKTDPELKLTDKILPVETDKKPQGKQLQTRADYLLKLLRKGLEKKGAVTGGEEAKLKKRKPRVKKENKVPRLKEEHGIELSSPRHSDNPSEEGEVKDDGLEKSPMKKKQKKKENKENKEKQMSSRKDKEGDKERKKSKDKKEKPKSGDAKSSSKSKRSQGPVHITAGSEPVPIGEDEDDDLDQETFSICKERMRPVKKALKQLDKPDKGLNVQEQLEHTRNCLLKIGDRIAECLKAYSDQEHIKLWRRNLWIFVSKFTEFDARKLHKLYKMAHKKRSQEEEEQKKKDDVTGGKKPFRPEASGSSRDSLISQSHTSHNLHPQKPHLPASHGPQMHGHPRDNYNHPNKRHFSNADRGDWQRERKFNYGGGNNNPPWGSDRHHQYEQHWYKDHHYGDRRHMDAHRSGSYRPNNMSRKRPYDQYSSDRDHRGHRDYYDRYAKGCETPGANLCQELFLGRK

SEQ ID NO: 3 - DNA, 20 bp, Artificial
  forward primer for amplifying mouse CHD2 gene
acgacgacgatgatgatgaa

SEQ ID NO: 4 - DNA, 20 bp, Artificial
  reverse primer for amplifying mouse CHD2 gene
ggctcctttctttcccagtc

SEQ ID NO: 5 - DNA, 21 bp, Artificial
  forward primer for amplifying human CHD2 gene
gcacaggacttcaaagcaaac

SEQ ID NO: 6 - DNA, 19 bp, Artificial
  reverse primer for amplifying human CHD2 gene
tgtccacttcctggatcac

SEQ ID NO: 7 - DNA, 18 bp, Artificial
  forward primer for amplifying canine CHD2 gene
caagaagaggaggagcaa

SEQ ID NO: 8 - DNA, 21 bp, Artificial
  reverse primer for amplifying canine CHD2 gene
gtggagtgtgggactgagata

SEQ ID NO: 9 - DNA, 17 bp, Artificial
  QuantiProbe for canine CHD2 gene
attggtggtaagaagcc

SEQ ID NO: 10 - DNA, 21 bp, Artificial
  forward primer for amplifying canine SCF gene
cagcagtagcagtaataggaa

SEQ ID NO: 11 - DNA, 19 bp, Artificial
  reverse primer for amplifying canine SCF gene
gctccaaaagcaaacccaa

SEQ ID NO: 12 - DNA, 17 bp, Artificial
  QuantiProbe for canine SCF gene
caacttacaatgggcag

SEQ ID NO: 13 - DNA, 19 bp, Artificial
  forward primer for amplifying canine c-kit gene
atagacccaacacagcttc

SEQ ID NO: 14 - DNA, 21 bp, Artificial
  reverse primer for amplifying canine c-kit gene
cagcacccaaagttttcccaa

SEQ ID NO: 15 - DNA, 17 bp, Artificial
  QuantiProbe for canine c-kit gene
tcccagaaacaggctga

SEQ ID NO: 16 - DNA, 18 bp, Artificial
  forward primer for amplifying canine GAPDH gene
ctggagaaagctgccaaa

SEQ ID NO: 17 - DNA, 18 bp, Artificial
  reverse primer for amplifying canine GAPDH gene
tgttgaagtcacaggaga

SEQ ID NO: 18 - DNA, 17 bp, Artificial
  QuantiProbe for canine GAPDH gene
agaaggtagtgaagcag